(19) United States
(12) Patent Application Publication
(10) Pub. No.: US 2021/0069227 A1
(43) Pub. Date: Mar. 11, 2021

(54) A3 ADENOSINE RECEPTOR LIGAND FOR MANAGING CYTOKINE RELEASE SYNDROME

(71) Applicant: CAN-FITE BIOPHARMA LTD, Petach Tikva (IL)

(72) Inventor: Pnina FISHMAN, Herzliya (IL)

(73) Assignee: CAN-FITE BIOPHARMA LTD, Petach Tikva (IL)

(21) Appl. No.: 16/647,282

(22) PCT Filed: Sep. 16, 2018
(86) PCT No.: PCT/IL2018/051034

§ 371 Date: Mar. 13, 2020

(30) Foreign Application Priority Data
Sep. 17, 2017 (IL) 254535

(51) Int. Cl. A61K 31/7076; A61K 31/4745

(52) U.S. Cl. CPC A61K 31/7076; A61K 31/4745

(57) ABSTRACT

Provided is a method of managing cytokine release syndrome (CRS) in a subject undergoing immunotherapy treatment, the method includes administering to the subject an amount of an A₃ adenosine receptor (A₃AR) ligand effective to manage one or more of (i) level of at least one inflammatory cytokine and (ii) at least one CRS symptom; wherein the management is without significantly affecting the immunotherapy treatment. Also provided is an A₃AR ligand and a composition including the ligand for use in the management of cytokine release syndrome (CRS) in a subject undergoing immunotherapy treatment, the management includes one or more of (i) managing level of at least one inflammatory cytokine and (ii) managing at least one CRS symptom; wherein the management is without significantly affecting the immunotherapy treatment.

### TECHNOLOGICAL FIELD

The present disclosure relates to immunotherapy, and particularly, to the therapeutic uses of A₃ adenosine receptor ligands for treating immunoocology complications.

### BACKGROUND ART

References considered to be relevant as background to the presently disclosed subject matter are listed below:
Daniel W. Lee et al. Current concepts in the diagnosis and management of cytokine release syndrome BLOOD 124(2):188-195 (2014)
Miller, Victoria, and Prasamsa Pandey. “Identifying And Treating Cytokine Release Syndrome.” ONCOLOGY NURSING FORUM. 44(2) 125 ENTERPRISE DR, PITTSBURGH, Pa. 15275 USA: ONCOLOGY NURSING SOC, 2017.

Acknowledgement of the above references herein is not to be inferred as meaning that these are in any way relevant to the patentability of the presently disclosed subject matter.

### BACKGROUND

Immunotherapy utilized the immune system mechanisms against cancer. One promising technique uses adoptive cell transfer (ACT), in which immune cells are modified to recognize and attack their tumors. One example of ACT is when a patient's own cytotoxic T-cells, or a donor's, are engineered to express a chimeric antigen receptor (CAR T-cells) targeted to a tumor specific antigen expressed on the surface of the tumor cells. However, overly stimulated immune activity resulting from such immunooncological treatments may lead to cytokine associated toxicity, referred to by the term Cytokine Release Syndrome (CRS) or cytokine storm.

To date, corticosteroids, biological therapies, such as anti-IL6 therapies and anti-inflammatory drugs are being evaluated to control cytokine release syndrome in patients administered with immunooncology drugs. However, steroids have the risk of affecting treatment and putting the patients in danger of sepsis and opportunistic infections. Anti-inflammatory drugs are less effective in controlling a very large number of pro-inflammatory cytokines as typical with CRS.

CRS is an increasing recognized risk involved in adoptive T-cell therapy and antibodies administration. Immunosuppression was found to be effective in treating CRS yet it conflicts with the initial aim of immunotherapy.

Daniel W. Lee et al. describe current concepts in the diagnosis and management of CRS. Among others. Daniel Lee et al. suggest a proactive management strategy that incorporates a grading system and treatment algorithm designed to administer early immunosuppression for patients at highest risk while avoiding unnecessary immunosuppression due to the potential risk of diminishing antitumor efficacy.

Miller, Victoria. and Prasamsa Pandey describe Chimeric antigen receptor (CAR) T-cell therapy as a promising tool for treatment of hematologic malignancies, and the adverse events following CAR T-cell infusion, including, predominantly, cytokine release syndrome (CRS). Miller, Victoria, and Prasamsa Pandey concentrate on grading system of CRS.

### GENERAL DESCRIPTION

In accordance with a first of its aspects, the present disclosure provides a method of managing cytokine release syndrome (CRS) in a subject undergoing immunotherapy treatment, particularly immunooncology treatment. The method comprises administering to the subject an amount of an A₃ adenosine receptor (A₃AR) ligand effective to manage one or more of (i) level of at least one inflammatory cytokine and (ii) at least one CRS symptom; wherein said management is without significantly affecting said immunotherapy treatment.

In accordance with a second of its aspects, the present disclosure provides an A₃AR ligand for use in the management of CRS in a subject undergoing immunotherapy treatment, said management comprises one or more of (i) managing level of at least one inflammatory cytokine and (ii) managing at least one CRS symptom; wherein said management is without significantly affecting said immunotherapy treatment.

Finally, in accordance with a third of its aspects, the present disclosure provides a pharmaceutical composition for the management of CRS in a subject undergoing immunotherapy treatment, the composition comprising a physiologically acceptable carrier and an a therapeutically effective amount of A₃AR ligand. The pharmaceutical composition that is indicated for the management of CRS in a subject undergoing immunotherapy treatment is typically accompanied by instruction for use of said composition in that indication.

### DETAILED DESCRIPTION OF EMBODIMENTS

The present disclosure is based on findings that A₃AR is effective in significantly reducing net levels of cytokines in subjects having a condition exhibited, inter alia, by elevated cytokines activation. The reduction of said net level is typically simultaneous for a variety of cytokine. The term “simultaneous” should be understood as that that may be observed for the variety of cytokines in repeated blood tests (in other words a repeated blood test will witness such a reduction across a variety of cytokines).

Specifically, it has been envisaged by the inventors, that this significant effect on cytokine net level may be employed to advantage of effectively managing the life-threatening cytokine release syndrome (CRS), also known as cytokine storm, manifested during immunotherapy treatment, especially, during immunooncology treatment.

Cytokine Release Syndrome (CRS) encompasses any cytokine release related toxicity, or any non-antigen specific toxicity deriving from a hypersensitive or hyper-reactive immune response. Hyper-reaction is considered as any change that differs from the change that would be observed in the patient prior to immunotherapy. At times, CRS is linked with the activation and\or release of lymphocytes such as B-cells T-cells, and natural killer (NK) cells and\or myeloid cells such as macrophages, dendritic cells, and monocytes. CRS was observed following therapeutic monoclonal antibody infusions such as anti-CD #, anti-CD52, anti-CD20 and TGN1412.

A severe form of CRS is known as cytokine storm and may, at times be a life-threatening condition. Typically, cytokine storm encompasses hyper release of inflammatory mediators in response to stimulation of T cells and macrophages by pathogens and immune insults. During a cytokine storm the level of interleukins (e.g. IL-1, IL-6, IL1), TNF-α, and other circulating mediators of inflammation, storms out of control.

Thus, disclosed herein are a method, use and composition for managing CRS in a subject undergoing immunotherapy treatment. The method, use and composition make use of an A₃AR ligand effective to manage one or more of (i) level of at least one inflammatory cytokine and (ii) at least one CRS symptom; this being without significantly affecting said immunotherapy treatment.

In the context of the present disclosure the term management or managing refers to the control an overall cytokine level in a subject undergoing immunotherapy treatment, without significantly compromising the effectiveness of the immunotherapy treatment. Preferably, the overall level of cytokines, including IL-1, IL-6, IL-8, TNF-alpha, MIP-1 α, MCP-1 will decrease after administration of the A₃AR ligand to a subject having a cytokine storm, or such so as to achieve a balance between the various pro-inflammatory cytokines that prevents or mitigates the toxic impact, mid or life threatening impact of the cytokine storm as was prior to said administration. At times, such balance would be manifested by a steady overall cytokine level comparable to the overall level of the cytokines prior to immunotherapy treatment and or before the development of a severe CRS even if not all cytokines' level have been similarly affected by the administration of the A₃AR ligand.

Specifically, the CRS management is in a subject undergoing immunotherapy. In the context of the present disclosure when referring to undergoing treatment it is to be understood as referring to a treatment where the administration of the A₃AR ligand can be prior to, following, or during the providing to the subject of the immunotherapeutic treatment. When a subject is undergoing a treatment under a treatment protocol that involves several administration of immunotherapeutic agents, ‘prior’ may refer to administration before the onset of the treatment protocol, i.e. the patient has not received yet any immunotherapeutic treatment, or it may refer to administration of said ligand prior to one or more administrations of the immunotherapeutic drug during an on-going immunotherapy treatment regimen (that may comprise several immunotherapeutic drug administrations over a treatment period, e.g. daily, weekly, monthly, etc.).

Similarly, administration of the ligand during the immunotherapy treatment may refer to administration of the A₃AR ligand simultaneously with the administration of the immunotherapeutic agent, or administration the A₃AR ligand in between two consecutive administrations of the immunotherapeutic agent; and administration of the ligand after the administration of the immunotherapeutic agent, may refer to the administration of the ligand after the administration of the immunotherapeutic agent or after the entire immunotherapy treatment protocol has ended.

When referring to immunotherapy, it is to be understood any medical intervention aimed at curing, preventing or mitigating a disease by providing a subject with an immunomodulatory treatment regimen that involves stimulating, enhancing or reducing (depending on the disease being treated) of the subject's immune system and response. In some embodiments, and without being limited thereto, immunotherapy includes an immunomodulatory regimen that involves administration of any one or combination of monoclonal antibodies, infusion of cells and\or applying adoptive T-cell therapy. e.g. monoclonal cell adoptive therapy.

In some embodiments, immunotherapy includes the utilization of chimeric antigen receptors (CARs) and/or chimeric T-cells (CAR-T).

In some embodiments, the immunotherapy is selected from Chimeric Antigen Receptor (CAR)-T cell Therapy, bispecific T cell-engaging antibodies, monoclonal antibody therapy, mononuclear cell adoptive immunotherapy and anti-PD-1 therapy.

In some embodiments, the immunotherapy is (CAR)-T cell Therapy.

CRS may be manifested during various types of treatment regimens for treating various diseases, for example cancer.

In the context of the present disclosure when referring to cancer it is to be understood as encompassing any cancerous condition for which immunotherapy treatment is found to be effective.

In some embodiments, the immunotherapy is for treating solid cancer. Smitha Menon et al. [Smitha Menon, Sarah Shin and Grace Dy, Cancers 2016, 8, 106] provide a review on the advances in cancer immunotherapy in solid Tumors and show that immunotherapy has already been proven as a potential remedy (some being under clinical trials) for various tumors such as melanoma, lung cancer, genitourinary (GU) cancers, malignant Mesothelioma, Merkel cells, colorectal cancers, hepatocellular carcinoma (HCC), Hodgkin's lymphoma. Others have described objective responses to immunotherapy of prostate cancer, kidney cancer, bladder cancer, ovarian cancer and others.

In some other embodiments, the caner is a hematological cancer. Without being limited thereto, when referring to a hematological cancer it encompasses any one of cancer associated with CD19 expression, such as B-cell acute lymphocytic leukemia (B-ALL), T-cell acute lymphocytic leukemia (T-ALL), acute lymphocytic leukemia (ALL), chronic myelogenous leukemia (CML), chronic lymphocytic leukemia (CLL), B-cell promyelocytic leukemia, blastic plasmacytoid dendritic cell neoplasm. Burkitt's lymphoma, diffuse large B cell lymphoma, GC (germinal center)-DLBCL, NGC (non-germinal center) -DLBCL, transformed FL, double hit DLBCL, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, and Waldenstrom macroglobulinemia.

In some embodiments, the cancer is chronic lymphocytic leukemia (CLL). CLL immunotherapies, e.g. T-Cell Engaging Therapies, are known to be involved with a cytokine activation profile, and with the risk of turning into a mid to life threatening state. Therefore, the use of A₃AR ligand for management of CLL may be of particular interest.

It is important to note that in the context of the present disclosure the CRS management is without significantly affecting the immunotherapeutic treatment. In other words, while affecting the level of cytokines involved in the cytokine storm, the desired therapeutic effect of the immunotherapeutic treatment on the target disease, e.g. cancer, is overall achieved by reducing, elimination, inhibiting, curing etc. of this target disease as a result of the immunotherapeutic treatment. While the term “without significantly affecting” has the meaning as generally describe above, in some embodiments, the administration of the A₃AR ligand does not statistically significantly compromise the immunotherapeutic treatment in terms of reduced or delayed effect of the immunotherapy treatment; or even if there is some degree of reduced or delayed effect, it is generally insignificant to the overall therapeutic effect.

In some embodiments, the terms “without significantly affecting” or “without significantly compromising the effectiveness” should be understood as meaning that the efficacy of the immunotherapy treatment, as managed by standard clinical measures of efficacy, either remains the same or is not reduced beyond 30%, typically not beyond 25%, 20%, 15%, or often also not beyond 10% below that that would have been observed in the absence of a treatment by an A₃AR ligand.

The effectiveness of the A₃AR ligand on CRS can be determined quantitatively, e.g. by measuring the level of one or more of the cytokines involved with the syndrome, e.g. cytokines having, before the A₃AR ligand administration a net level that is statistically significantly above or below a baseline or a reference level and their modulation is, thus, desired in order to mitigate the CRS symptoms. A baseline level or reference level may be the level of the one or more cytokines in the subject before the immunotherapy treatment was initiated, or a level determined from an appropriate group of healthy subjects (e.g. an average level of subjects of same age, gender, etc.). The level of the cytokine can be determined from a subject's peripheral blood sample.

Cytokines that are typically involved with CRS, and the level of which can be used to determine effectiveness of administration of A₃AR ligand are pro-inflammatory cytokines. The cytokines involved in CRS are any one or combination of Interleukin-1 (IL-1), IL-6, IL-13. Interferon-gamma (INF-γ). Tumor Necrosis Factor alpha (TNF-α), Macrophage Inflammatory Protein 1 alpha (MIP1α), Glycoprotein 130 (gp130), eotaxin and Monocyte chemoattractant protein-1 (MCP-1).

In some embodiments, the beneficial effect may be determined (qualitatively or quantitatively) through alleviation of one or more symptoms of CRS. Without being limited thereto, the CRS symptoms include any one or combination of fatigue, fever, nausea, vomiting, headache, rash, diarrhea, tachypnea, hypoxemia, tachycardia, widened pulse pressure, hypotension, increased cardiac output, potentially diminished cardiac output, elevated D-dimer, hypofibrinogenemia, azotemia, transaminitis, hyperbilirubinemia, confusion, delirium, word finding difficulty, frank aphasia, hallucinations, tremor, dymetria, altered gait, seizures and combination of any of the above. According to some embodiments the CRS symptom is determined according to the grading system of the National Cancer Institute Common Terminology Criteria for Adverse Events (CTCAE v4.0).

When referring to an A₃ adenosine receptor ligand or A₃AR ligand it is to be understood to mean any compound capable of directly (e.g. via the receptor binding site) or indirectly (e.g. via an allosteric binding site) modulating the activity of the A₃ adenosine receptor, this including full or partial activation of the A₃ adenosine receptor. The A₃AR ligand is thus a molecule that exerts its prime effect through the enhancement of the activity of the A₃AR irrespective of whether the activation is via the binding site or allosteric binding site. This means that at the doses it is being administered it essentially affects only the A₃AR.

In some embodiments, the A₃AR ligand encompasses A₃AR agonists or A₃AR allosteric enhancers.

When referring to an A₃AR agonist it is to be understood to mean any ligand capable of specifically binding to the A₃ adenosine receptor, thereby fully or partially activating the A₃ adenosine receptor. The A₃AR agonist is thus a molecule that exerts its prime effect through the binding and activation of the A₃AR. This means that at the doses it is being administered it essentially binds to and activates only the A₃AR.

However, it should be understood that some A₃AR agonists can also interact with and activate other receptors, however, with lower affinities.

A molecule will be considered an A₃AR agonist in the context of the present disclosure (namely a molecule that exerts its prime effect through the binding and activation A₃AR) if its affinity to the A₃AR is at least 3 times (i.e. its Ki to the A₃AR is at least 3 times lower), preferably 10 times, desirably 20 times and most preferably at least 50 times larger than the affinity to any other of the adenosine receptors (i.e. A₁, A₂ₐ and A_{2b}).

The affinity of an A₃AR agonist to the human A₃AR as well as its relative affinity to the other human adenosine receptors can be determined by a number of assays, such as a binding assay. Examples of binding assays include providing membranes containing a receptor and measuring the ability of the A₃AR agonist to displace a bound radioactive agonist; utilizing cells that display the respective human adenosine receptor and measuring, in a functional assay, the ability of the A₃AR agonist to activate or deactivate, as the case may be, downstream signaling events such as the effect on adenylate cyclase measured through increase or decrease of the cAMP level; etc. Clearly, if the administered level of an A₃AR agonist is increased such that its blood level reaches a level approaching that of the Ki of the A₁, A₂ₐ and A_{2b} adenosine receptors, activation of these receptors may occur following such administration, in addition to activation of the A₃AR. An A₃AR agonist is thus preferably administered at a dose such that the blood level is such so that essentially only the A₃AR will be activated.

In some embodiments, the A₃AR agonist is a molecule that has a purine backbone. In some embodiment, the purine containing compound may be determined as an A₃AR agonist based on acceptable structure-function activity assays.

The characteristic of some A₃AR agonists and methods of their preparation are described in detail in, inter alia, U.S. Pat. Nos. 5,688,774; 5,773,423, 5,573,772, 5,443,836, 6,048,865, WO 95/02604, WO 99/20284. WO 99/06053, WO 97/27173 and WO 01/19360, all of which are incorporated herein by reference.

According to some embodiments of the present disclosure, the A₃AR agonist is a purine derivative falling within the scope of the general formula (I):

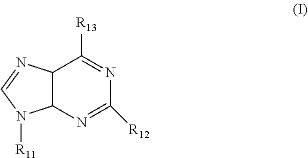

wherein,
R₁₁ represents an alkyl, hydroxyalkyl, carboxyalkyl or cyanoalkyl or a group of the following general formula (II):

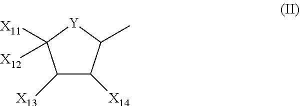

in which:
Y represents oxygen, sulfur or CH₂;
X₁₁ represents H, alkyl. R^{e}R^{f}NC(═O)— or HOR^{g}—, wherein
   R^{e} and R^{f} may be the same or different and are selected from the group consisting of hydrogen, alkyl, amino, haloalkyl, aminoalkyl, BOC-aminoalkyl, and cycloalkyl or are joined together to form a heterocyclic ring containing two to five carbon atoms; and
   R^{g} is selected from the group consisting of alkyl, amino, haloalkyl, aminoalkyl, BOC-aminoalkyl, and cycloalkyl;
X₁₂ is H, hydroxyl, alkylamino, alkylamido or hydroxyalkyl;
X₁₃ and X₁₄ represent independently hydrogen, hydroxyl, amino, amido, azido, halo, alkyl, alkoxy, carboxy, nitrilo, nitro, trifluoro, aryl, alkaryl, thio, thioester, thioether, —OCOPh, —OC(═S)OPh or both X₁₃ and X₁₄ are oxygens connected to >C═S to form a 5-membered ring, or X₁₂ and X₁₃ form the ring of formula (III):

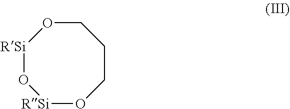

where R′ and R″ represent independently an alkyl group;
R₁₂ is selected from the group consisting of hydrogen, halo, alkylether, amino, hydrazido, alkylamino, alkoxy, thioalkoxy, pyridylthio, alkenyl; alkynyl, thio, and alkylthio; and
R₁₃ is a group of the formula —NR₁₅R₁₆ wherein
R₁₅ is a hydrogen atom or a group selected from alkyl, substituted alkyl or aryl-NH—C(Z)—, with Z being O, S, or NR^{a} with R^{e} having the above meanings; wherein when R₁₅ is hydrogen than
R₁₆ is selected from the group consisting of R- and S-1-phenylethyl, benzyl, phenylethyl or anilide groups unsubstituted or substituted in one or more positions with a substituent selected from the group consisting of alkyl, amino, halo, haloalkyl, nitro, hydroxyl, acetoamido, alkoxy, and sulfonic acid or a salt thereof; benzodioxanemethyl, fururyl, L-propylalanyl-aminobenzyl, β-alanylamino-benzyl, T-BOC-β-alanylaminobenzyl, phenylamino, carbamoyl, phenoxy or cycloalkyl; or R₁₆ is a group of the following formula (IV):

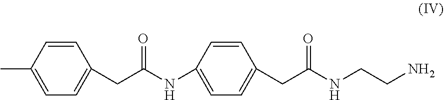 [SMILES: Cc1ccc(CC(=O)Nc2ccc(CC(=O)NCCN)cc2)cc1]

or when R₁₅ is an alkyl or aryl-NH—C(Z)—, then, R₁₆ is selected from the group consisting of heteroaryl-NR^{a}—C(Z)—, heteroaryl-C(Z)—, alkaryl-NR^{a}—C(Z)—, alkaryl-C(Z)—, aryl-NR—C(Z)— and aryl-C(Z)—; Z representing an oxygen, sulfor or amine.

Exemplary A₃AR agonist (disclosed in U.S. Pat. No. 5,688,774 at column 4, lines 67-column 6, line 16: column 5, lines 40-45; column 6, lines 21-42; column 7, lines 1-11: column 7, lines 34-36; and column 7, lines 60-61):
N⁶-(3-iodobenzyl)-9-methyladenine;
N⁶-(3-iodobenzyl)-9-hydroxyethyladenine;
R—N⁶-(3-iodobenzyl)-9-(2,3-dihydroxypropyl)adenine:
S—N⁶-(3-iodobenzyl)-9-(2,3-dihydroxypropyl)adenine;
N⁶-(3-iodobenzyladenin-9-yl)acetic acid;
N⁶-(3-iodobenzyl)-9-(3-cyanopropyl)adenine;
2-chloro-N⁶-(3-iodobenzyl)-9-methyladenine;
2-amino-N⁶-(3-iodobenzyl)-9-methyladenine;
2-hydrazido-N⁶-(3-iodobenzyl)-9-methyladenine;
N⁶-(3-iodobenzyl)-2-methylamino-9-methyladenine;
2-dimethylamino-N⁶-(3-iodobenzyl)-9-methyladenine;
N⁶-(3-iodobenzyl)-9-methyl-2-propylaminoadenine;
2-hexylamino-N⁶-(3-iodobenzyl)-9-methyladenine;
N⁶-(3-iodobenzyl)-2-methoxy-9-methyladenine;
N⁶-(3-iodobenzyl)-9-methyl-2-methylthioadenine;
N⁶-(3-iodobenzyl)-9-methyl-2-(4-pyridylthio)adenine;
(1S, 2R, 3S, 4R)-4-(6-amino-2-phenylethylamino-9H-purin-9-yl)cyclopentane-1,2,3-triol;
(1S, 2R, 3S, 4R)-4-(6-amino-2-chloro-9H-purin-9-yl) cyclopentane-1,2,3-triol;
(±)-9-[2α,3α-dihydroxy-4β-(N-methylcarbamoyl)cyclopent-1β-yl)]-N⁶-(3-iodobenzyl)-adenine;
2-chloro-9-(2′-amino-2′,3′-dideoxy-β-D-5′-methyl-arabino-furonamido)-N⁶-(3-iodobenzyl)adenine;
2-chloro-9-(2′,3′-dideoxy-2′-fluoro-β-D-5′-methyl-arabino furonamido)-N⁶-(3-iodobenzyl)adenine;
9-(2-acetyl-3-deoxy-β-D-5-methyl-ribofuronamido)-2-chloro-N⁶-(3-iodobenzyl)adenine;
2-chloro-9-(3-deoxy-2-methanesulfonyl-β-D-5-methyl-ribofuronamido)-N⁶-(3-iodobenzyl)adenine;
2-chloro-9-(3-deoxy-β-D-5-methyl-ribofuronamido)-N⁶-(3-iodobenzyl)adenine;
2-chloro-9-(3,5-1,1,3,3-tetraisopopyldisiloxyl-β-D-5-ribofuranosyl)-N⁶-(3-iodobenzyl)adenine;
2-chloro-9-(2′,3′-O-thiocarbonyl-β-D-5-methyl-ribofunamido)-N⁶-(3-iodobenzyl)adenine;
9-(2-phenoxythiocarbonyl-3-deoxy-β-D-5-methyl-ribofuronamido)-2-chloro-N⁶-(3-iodobenzyl)adenine;
1-(6-benzylamino-9H-purin-9-yl)-1-deoxy-N,4-dimethyl-β-D-ribofuranosiduronamide;
2-chloro-9-(2,3-dideoxy-β-D-5-methyl-ribofuronamido)-N⁶ benzyladenine;
2-chloro-9-(2′-azido-2′,3′-dideoxy-β-D-5′-methyl-arabino-furonamido)-N⁶-benzyladenine;
2-chloro-9-(β-D-erythrofuranoside)-N⁶-(3-iodobenzyl)adenine;
N⁶-(benzodioxanemethyl)adenosine;
1-(6-furfurylamino-9H-purin-9-yl)-1-deoxy-N-methyl-β-D-ribofuranosiduronamide;
N⁶-[3-(L-prolylamino)benzyl]adenosine-5′-N-methyluronamide;
N⁶-[3-(P-alanylamino)benzyl]adenosine-5′-N-methyluronamide;
N⁶-[3-(N-T-Boc-β-alanylamino)benzyl]adenosine-5′-N-methyluronamide
6-(N′-phenylhydrazinyl)purine-9-β-ribofuranoside-5′-N-methyluronamide;
6-(O-phenylhydroxylamino)purine-9-β-ribofuranoside-5′-N-methyluronamide;
9-(β-D-2′,3′-dideoxyerythrofuranosyl)-N⁶-[(3-β-alanylamino)benzyl]adenosine;
9-(β-D-erythrofuranoside)-2-methylamino-N⁶-(3-iodobenzyl)adenine;
2-chloro-N-(3-iodobenzyl)-9-(2-tetrahydrofuryl)-9H-purin-6-amine;
2-chloro-(2′-deoxy-6′-thio-L-arabinosyl)adenine; and
2-chloro-(6′-thio-L-arabinosyl)adenine.

In some embodiments, the A₃AR agonists, are a compound already disclosed in U.S. Pat. No. 5,773,423, and is a compound of the formula (V):

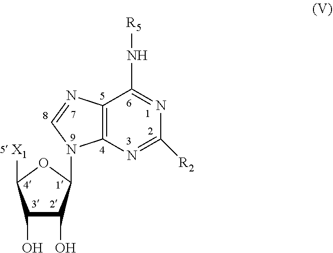

wherein

X₁ is R^{a}R^{b}NC(═O), wherein R^{a} and R^{b} may be the same or different and are selected from the group consisting of hydrogen, C₁-C₁₀ alkyl, amino, C₁-C₁₀ haloalkyl, C₁-C₁₀ aminoalkyl, and C₃-C₁₀ cycloalkyl;

R₂ is selected from the group consisting of hydrogen, halo, C₁-C₁₀ alkyoxy, amino, C₂-C₁₀ alkenyl, and C₂-C₁₀ alkynyl; and

R₅ is selected from the group consisting of R- and S-1-phenylethyl an unsubstituted benzyl group, and a benzyl group substituted in one or more positions with a substituent selected from the group consisting of C₁-C₁₀ alkyl, amino, halo, C₁-C₁₀ haloalkyl, nitro, hydroxy, acetamido, C₁-C₁₀ alkoxy, and sulfo.

More specific compounds include those of the above formula wherein R^{a} and R^{b} may be the same or different and are selected from the group consisting of hydrogen and C₁-C₁₀ alkyl, particularly when R₂ is hydrogen or halo, especially hydrogen.

Additional specific compounds are those compounds wherein R^{a} is hydrogen and R₂ is hydrogen, particularly when R₅ is unsubstituted benzyl.

More specific compounds are such compounds wherein R^{b} is a C₁-C₁₀ alkyl or C₃-C₁₀ cycloalkyl, particularly a C₁-C₁₀ alkyl, and more particularly methyl.

Especially specific are those compounds where R^{a} is hydrogen, R^{b} is C₁-C₁₀ alkyl or C₃-C₁₀ cycloalkyl, and R₅ is R- or S-1-phenylethyl or a benzyl substituted in one or more positions with a substituent selected from the group consisting of halo, amino, acetamido, C₁-C₁₀ haloalkyl, and sulfo, where the sulfo derivative is a salt, such as a triethylammonium salt.

An example of an especially preferred compound disclosed in U.S. Pat. No. 5,773,423 is N⁶-(3-iodobenzyl)-adenosine-5′-N-methyluronamide (IB-MECA, also referred to at times as CF101).

Another example of an especially preferred compound disclosed in U.S. Pat. No. 5,773,423 is 2-Chloro-N⁶-(3-iodobenzyl)-adenosine-5′-N-methyluronamide (Cl-IB-MECA, also referred to at times as CF102).

In addition, those compounds in which R₂ is a C₂-C₁₀ alkenylene of the formula R^{d}—C═C— where R^{d} is a C₁-C₈ alkyl are also particularly noted in U.S. Pat. No. 5,773,423.

Also specific are those compounds wherein R₂ is other than hydrogen, particularly those wherein R₂ is halo, C₁-C₁₀ alkylamino, or C₁-C₁₀ alkylthio, and, more preferably, when additionally R^{a} is hydrogen, R^{b} is a C₁-C₁₀ alkyl, and/or R is a substituted benzyl.

Further exemplary A₃AR agonists disclosed in U.S. Pat. No. 5,773,423 are modified xanthine-7-ribosides having the formula (VI):

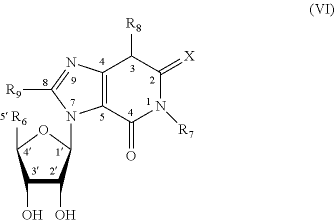

wherein

X is O;

R₆ is R^{a}R^{b}NC(═O), wherein R^{a} and R^{b} may be the same or different and are selected from the group consisting of hydrogen, C₁-C₁₀ alkyl, amino, C₁-C₁₀ haloalkyl, C₁-C₁₀ aminoalkyl, and C₃-C₁₀ cycloalkyl:

R₇ and R₈ may be the same or different and are selected from the group consisting of C₁-C₁₀ alkyl, R- and S-1-phenylethyl, an unsubstituted benzyl group, and a benzyl group substituted in one or more positions with a substituent selected from the group consisting of C₁-C₁₀ alkyl, amino, halo, C₁-C₁₀ haloalkyl, nitro, hydroxy, acetamido, C₁-C₁₀ alkoxy, and sulfo; and

R₉ is selected from the group consisting of halo, benzyl, phenyl, and C₃-C₀ cycloalkyl.

WO 99/06053 discloses in examples 19-33 compounds selected from:
N⁶-(4-biphenyl-carbonylamino)-adenosine-5′-N-ethyluronamide;
N⁶-(2,4-dichlorobenzyl-carbonylamino)-adenosine-5′-N-ethyluronamide;
N⁶-(4-methoxyphenyl-carbonylamino)-adenosine-5′-N-ethyluronamide;
N⁶-(4-chlorophenyl-carbonylamino)-adenosine-5′-N-ethyluronamide;
N⁶-(phenyl-carbonylamino)-adenosine-5′-N-ethyluronamide;
N⁶-(benzylcabamoylamino)-adenosine-5′-N-ethyluronamide;
N⁶-(4-sulfonamido-phenylcarbamoyl)-adenosine-5′-N-ethyluronamide;
N⁶-(4-acetyl-phenylcarbamoyl)-adenosine-5′-N-ethyluronamide;
N⁶—((R)-α-phenylethylcarbamoyl)-adenosine-5′-N-ethyluronamide;
N⁶—((S)-α-phenylethylcarbamoyl)-adenosine-5′-N-ethyluronamide;
N⁶-(5-methyl-isoxazol-3-yl-carbamoyl)-adenosine-5′-N-ethyluronamide;
N⁶-(1,3,4-thiadiazol-2-yl-carbamoyl)-adenosine-5′-N-ethyluronamide;
N⁶-(4-n-propoxy-phenylcarbamoyl)-adenosine-5′-N-ethyluronamide;
N⁶-bis-(4-nitophenylcarbamoyl)-adenosine-5′-N-ethyluronamide; and
N⁶-bis-(5-chloro-pyridin-2-yl-carbamoyl)-adenosine-5′-N-ethyluronamide.

When referring to A₃AR allosteric enhancer it is to be understood as referring to a ligand imparting a positive regulation, activation or incense of the receptor activity by binding at the receptor's allosteric site which may be different from the binding site of the endogenous ligand or agonist thereof. In this context, an enhancement is to be understood as denoting an effect of the ligand on the receptor exhibited by an increase of at least 15% in the efficacy of the A₃ adenosine receptor by binding of the ligand to the receptor's allosteric binding site and/or in a decrease in dissociation rate of adenosine or an A₃AR ligand to the orthosteric binding site.

In some embodiments the A₃AR enhancer, or imidazoquinoline derivative having affinity to the A₃AR has the following general formula (VII):

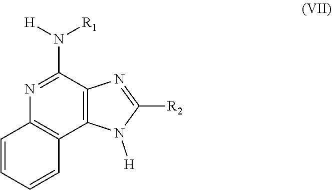

wherein:
R₁ represents an aryl or alkaryl being optionally substituted at the aromatic ring once or more with a substituent selected C₁-C₁₀ alkyl, halo, C₁-C₁₀ alkanol, hydroxyl, C₁-C₁₀ acyl, C₁-C₁₀ alkoxyl; C₁-C₁₀-alkoxycarbony, C₁-C₁₀ alkoxylalkyl; C₁-C₁₀ thioalkoxy; C₁-C₁₀ alkylether, amino, hydrazido, C₁-C₁₀ alkylamino, pyridylthio, C₂-C₁₀ alkenyl; C₂-C₁₀ alkynyl, thio, and C₁-C₁₀ alkylthio, acetoamido, sulfonic acid; or said substituents can form together a cycloalkyl or cycloalkenyl fused to said aryl, the cycloalkyl or cycloalkenyl optionally comprising one or more heteroatoms: provided that said aryl is not an unsubstituted phenyl group;
R₂ represents hydrogen or a substituent selected from C₁-C₁₀ alkyl, C₂-C₁₀ alkenyl; C₂-C₁₀ alkynyl, C₄-C₁₀ cycloalkyl, C₄-C₁₀ cycloalkenyl or a five to seven membered heterocyclic aromatic ring, C₅-C₁₅ fused cycloalkyl, bicyclic aromatic or heteroaromatic rings; or C₁-C₁₀ alkylether, amino, hydrazido, C₁-C₁₀ alkylamino, C₁-C₁₀ alkoxy, C₁-C₁₀-alkoxycarbony, C₁-C₁₀ alkanol, C₁-C₁₀ acyl, C₁-C₁₀ thioalkoxy, pyridylthio, thio, and C₁-C₁₀ alkylthio, acetoamido, sulfonic acid;

and pharmaceutically acceptable salts thereof.

According to some embodiments, the R₁ substituent in the A₃AR enhancer has the following general formula (VIII):

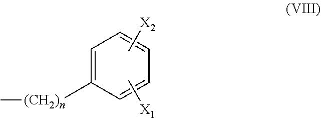

wherein n is 0 or an integer selected from 1-5; preferably, n is 0, 1 or 2; and
X₁ and X₂ which may be the same or different, are selected from hydrogen halogen, alkyl, alkanol or alkoxy, indanyl, pyrroline provided that when said n is 0, X₁ and X₂ are not hydrogen.

In yet some further embodiments. R₁ in A₃AR enhancer is a substituent having the above formula (VIII), wherein X₁ or X₂, which may be the same or different, are selected from hydrogen, chloro, methoxy, methanol or a substituent having the formulae (VIIIa) or (VIIIb):

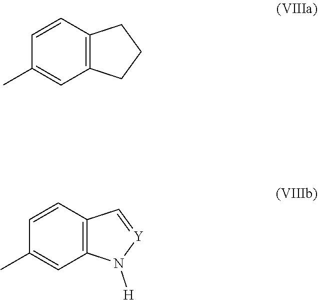

wherein Y is selected from N or CH.

In some yet further embodiments R₂ in A₃AR enhancer is selected from H, C₁₋₁₀ alkyl, C₄₋₁₀ cycloalkyl, the alkyl chain may be a straight or branched or form a four to seven membered cycloalkyl ring.

In yet some further embodiments, R₂ in A₃AR enhancer is selected from a five to seven membered heterocyclic aromatic ring.

In some embodiments, R₂ substituents in A₃AR enhancer are selected from H, n-pentyl, or a five membered heterocyclic aromatic ring having the following formula (IX):

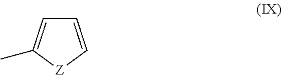

wherein Z is selected from O, S or NH, preferably O.

In accordance with some embodiments R₂ in A₃AR enhancer comprises one or more fused rings, particularly so as to form bicyclic substituents.

Non-limiting examples of bicyclic compounds which may be used to form the substituents in the context of the invention comprise bicyclo[2.2.1]heptane, bicyclo[4.1.0]heptane, bicyclo[4.1.0]heptan-3-carboxylic acid, bicyclo[3.1.0]hexan-3-carboxylic acid, bicyclo[4.1.0]heptan-2-carboxylic acid, bicyclo[3.1.0]hexan-2-carboxylic acid, and bicyclo[2.2.1]heptan-2-carboxylic acid.

In accordance with yet some other embodiments, R₂ in A₃AR enhancer may be selected from 2-cyclohexene and 3-cyclohexene.

Specific imidazoquinoline derivatives which may be used as allosteric effectors of the A₃AR are listed below:
N-(4-Methyl-phenyl)-2-cyclopentyl-1H-imidazo[4,5-c]quinolin-4-amine
N-(4-Methoxy-phenyl)-2-cyclopentyl-1H-imidazo[4,5-c]quinolin-4-amine
N-(3,4-Dichloro-phenyl)-2-cyclopentyl-1H-imidazo[4,5-c]quinolin-4-amine
N-(4-Chloro-phenyl)-2-cyclopentyl-1H-imidazo[4,5-c]quinolin-4-amine
N-(3-Methanol-phenyl)-2-cyclopentyl-1H-imidazo[4,5-c]quinolin-4-amine
N-([3,4-c]Indan)-2-cyclopentyl-1H-imidazo[4,5-c]quinolin-4-amine
N-(1H-indazol-6-yl)-2-cyclopentyl-1H-imidazo[4,5-c]quinolin-4-amine
N-(4-Methoxy-benzyl)-2-cyclopentyl-1H-imidazo[4,5-c]quinolin-4-amine
N-(1H-Indol-6-yl)-2-cyclopentyl-1H-imidazo[4,5-c]quinolin-4-amine
N-(Benzyl)-2-cyclopentyl-1H-imidazo[4,5-c]quinolin-4-amine
N-(Phenylethyl)-2-cyclopentyl-1H-imidazo[4,5-c]quinolin-4-amine
N-(3,4-Dichloro-phenyl)-2-cycloheptyl-1H-imidazo[4,5-c]quinolin-4-amine
N-(3,4-Dichloro-phenyl)-2-furyl-1H-imidazo[4,5-c]quinolin-4-amine
N-(3,4-Dichloro-phenyl)-2-cyclobutyl-1H-imidazo[4,5-c]quinolin-4-amine
N-(3,4-Dichloro-phenyl)-2-cyclohexyl-1H-imidazo[4,5-c]quinolin-4-amine
N-(3,4-Dichloro-phenyl)-2--1H-imidazo[4,5-c]quinolin-4-amine
N-(3,4-Dichloro-phenyl)-2-pentyl-1H-imidazo[4,5-c]quinolin-4-amine.

The above imidazoquinoline derivatives are regarded as allosteric enhancers as they were shown to have, on the one hand, reduced affinity, if any, to the orthosteric binding sites of the A₁ and A_{2A}, A_{2B} adenosine receptors and reduced affinity to the orthosteric binding site of the A₃ adenosine receptor, and on the other hand, high affinity to the allosteric site of the A₃ adenosine receptor (International Patent Application No.

WO07/089507, incorporated herein by reference).

In some embodiments, said A₃AR allosteric enhancer is an imidazoquinoline derivative selected from the group consisting of:
N-(3,4-Dichloro-phenyl)-2-cyclopentyl-1H-imidazo[4,5-]quinolin-4-amine;
N-(3,4-Dichloro-phenyl)-2-cycloheptyl-1H-imidazo[4,5-c]quinolin-4-amine;
N-(3,4-Dichloro-phenyl)-2-cyclobutyl-1H-imidazo[4,5-c]quinolin-4-amine; and
N-(3,4-Dichloro-phenyl)-2-cyclohexyl-1H-imidazo[4,5-c]quinolin-4-amine.

A specifically imidazoquinoline derivative in accordance with the present disclosure is N-(3,4-Dichloro-phenyl)-2-cyclohexyl-1H-imidazo[4,5-c]quinolin-4-amine (also referred to at times by the abbreviation LUF6000 or CF602), being an allosteric enhancer.

In the context of the general formulae disclosed herein above, the following meaning for the various terms is to be considered:

The term “alkyl” is used herein to refer to a linear or branched hydrocarbon chain having from 1 to 10 carbon atoms and more preferably 1 to 6 carbon atoms including, but not limited to, methyl, ethyl, n-propyl, isopropyl, n-butyl, t-butyl, n-heptyl, octyl and the like.

Similarly, the terms “alkenyl” and “alkynyl” denote a linear or branched hydrocarbon chain having, respectively, from 2 to 10, or from 3 to 10 carbon atoms and more preferably 2 to 6 or 3 to 6 carbon atoms, the alkenyl or alkynyl having at least one unsaturated bond.

The alkyl, alkenyl or alkynyl substituents may be substituted with a heteroatom containing group. Thus, it should be understood that while not explicitly stated, any of the alkyl modifications defined hereinabove and below, such as alkylthio, alkoxy, akanol, alkylamine etc, also include the corresponding alkenyl or alkynyl modifications, such as, akenylthio, akenyloxy, alkenol, alkenylamine, or respectively, akynylthio, alkynyloxy, alkynol, alkynylamine.

The term “aryl” denotes an unsaturated aromatic carbocyclic group of from 5 to 14 carbon atoms having a single ring (e. g., phenyl) or multiple condensed rings (e. g., naphthyl or anthryl). Preferred aryls include phenyl, indanyl, benzimidazole.

The term “alkaryl” refers to -alkylene-aryl groups preferably having from 1 to 10 carbon atoms in the alkylene moiety and from 6 to 14 carbon atoms in the aryl moiety.

Such alkaryl groups are exemplified by benzyl, phenethyl and the like.

The term “Substituted aryl” refers to an aromatic moiety which is substituted with from 1 to 3 substituents as defined above. A variety of substituents are possible, as appreciated by those versed in the art. Nonetheless, some preferred substituents include, without being limited thereto, halogen. (substituted) amino, nitro, cyano, alkyl, alkoxy, acyloxy or alkanol, sulphonyl, sulphynyl.

The term “Halo” or “halogen” refers to fluoro, chloro, bromo and iodo, preferably to chloro.

The term “acyl” refers to the groups H—C(O)— as well as alkyl-C(O)—.

The term “alkanol” refers to the group —COH as well as alk-OH, “alk” denoting an alkylene, alkenylene or alkynylene chain.

The term “alkoxy” is used herein to mean —O-alkyl, including, but not limited to, methoxy, ethoxy, propoxy, isopropoxy, n-butoxy and the like.

The term “alkylthio” is used herein to mean —S-alkyl, including, but not limited to, methylthio, ethylthio, n-propylthio, isopropylthio, n-butylthio and the like.

The term “alkoxyalkyl” is used herein to mean -alkyl-O-alkyl, including, but not limited to, methoxymethyl, ethoxymethyl, n-propoxymethyl, isopropoxymethyl, n-butoxymethyl, isobutoxymethyl, t-butoxymethyl and the like.

The term “cycloalkyl” is used herein to mean cyclic hydrocarbon radicals including, but not limited to, cyclopropyl, cyclobutyl, cyclopentyl, cyclohexyl, cycloheptyl and the like.

The term “alkoxycarbonyl” is used herein to mean —C(O)-alkyl, including, but not limited to, methoxycarbonyl, ethoxycarbonyl, propoxycarbonyl and the like.

The term “fused cycloalkyl” is used herein to mean any compound or substituent comprising at least two aliphatic rings which are connected at a single atom (to form a spirocyclic moiety), at two mutually bonded atoms or across a sequence of atoms (bridgehead). The fused rings may include any bicyclic, tricyclic as well as polycyclic moieties. Bicyclic substituents are preferred in accordance with some embodiments of the present disclosure.

The present disclosure also encompasses physiologically acceptable salts of any disclosed or defined A₃AR ligand, such as the above disclosed compounds. A physiologically acceptable salt refers to any non-toxic alkali metal, alkaline earth metal, and ammonium salt commonly used in the pharmaceutical industry, including the sodium, potassium, lithium, calcium, magnesium, barium ammonium and protamine zinc salts, which are prepared by methods known in the art. The term also includes non-toxic acid addition salts, which are generally prepared by reacting the ligand with a suitable organic or inorganic acid. The acid addition salts are those which retain the biological effectiveness and qualitative properties of the free bases and which are not toxic or otherwise undesirable. Examples include, inter alia, acids derived from mineral acids, hydrochloric, hydrobromic, sulfuric, nitric, phosphoric, metaphosphoric and the like. Organic acids include, inter alia, tartaric, acetic, propionic, citric, malic, malonic, lactic, fumaric, benzoic, cinnamic, mandelic, glycolic, gluconic, pyruvic, succinic salicylic and arylsulphonic, e.g. p-toluenesulphonic, acids.

The A₃AR ligand can be administered to the subject in any route available. For administration the ligand is typically combined with a physiologically acceptable carrier. The carrier may, at times, have the effect of the improving the delivery or penetration of the A₃AR ligand to the target tissue, for improving the stability of the A₃AR ligand, for slowing clearance rates, for imparting slow release properties, for reducing undesired side effects etc. The carrier may also be a substance that stabilizes the formulation (e.g. a preservative), for providing the formulation with an edible flavor, etc. The carriers may be any of those conventionally used and is limited only by chemical-physical considerations, such as solubility and lack of reactivity with the A₃AR ligand, and by the route of administration. The carrier may include additives, colorants, diluents, buffering agents, disintegrating agents, moistening agents, preservatives, flavoring agents, and pharmacologically compatible carriers. In addition, the carrier may be an adjuvant, which, by definition are substances affecting the action of the A₃AR ligand in a predictable way.

In some embodiments, the A₃AR and a carrier are formulated for oral administration. An oral formulation may be in the form of a pill, capsule, in the form of a syrup, emulsion, an aromatic powder, and other various forms Typical examples of carriers suitable for oral administration comprise (a) suspensions or emulsions in an appropriate liquid such as Cremophor RH40, or methylcellulose (e.g. Methocel A4M Premium); (b) capsules (e.g. the ordinary hard- or soft-shelled gelatin type containing, for example, surfactants, lubricants, and inert fillers), tablets, lozenges (wherein the active substance is in a flavor, such as sucrose and acacia or tragacanth or the active substance is in an inert base, such as gelatin and glycerin), and troches, each containing a predetermined amount of the tragacanth as solids or granules; (c) powders; (d) solution, typically when combined with a solubilizing enhancing agent; (e) liposome formulation; and others.

One non-limiting example for an oral administration form of the A₃AR ligand, IB-MECA includes the following ingredients and amounts formulated in the form of tablets:

TABLE 1
IB-MECA Tablets

| | Ingredient | Amount (mg) |
|---|---|---|
| Intragranular | IB-MECA | 1.000 |
| | Pregelatinized Starch | 10.00 |
| | Croscarmellose Sodium | 2.000 |
| | Lactose Monohydrate 310 | 64.25 |
| | Microcrystalline Cellulose | 20.00 |
| Extragranular | Croscarmellose Sodium | 2.000 |
| | Magnesium Stearate | 0.7500 |
| | Total | 100.00 |
| Coating | Opadry White | 3.000 |
| | Total | 103.0 |

In some embodiments, the A₃AR ligand can be formulated for administration as a nasal spray, and others.

Irrespective of the mode of administration, application of the ligand. i.e. treatment, may be acute treatment, e.g. upon manifestation of CRS, or long term treatment that includes a treatment regimen with the ligand.

In some embodiments, the A₃AR ligand is formulated and administered in an amount sufficient to provide a statistically significant (preferably P value <0.05) net change in the level of the at least one inflammatory cytokine as compared to a baseline level or reference level of the at least one inflammatory cytokine. When referring to level of cytokines in CRS, particularly in patients with cancer, it is particularly important to refer to fold change (e.g. fold increase), net change (e.g. net increase), or rate of change as baseline inflammatory cytokine levels which can be very high due to their underlying disease. These measurements will typically provide better correlation between the CRS severity than absolute cytokine levels. This is particularly relevant when the diagnostics require a profile of several different cytokines rather than changes in only 1 level.

The effective amount or amount sufficient to can readily be determined, in accordance with the invention, by administering to a plurality of tested subjects various amounts of the A₃AR ligand and then plotting the response (for example combining several beneficial effects) as a function of the amount. At times, the amount to be used may depend on a variety of factors such as mode of administration, age, weight, body surface area, gender, health condition and genetic factors of the subject; other administered drugs; etc.

As used herein, the forms “a”, “an” and “the” include singular as well as plural references unless the context clearly dictates otherwise. For example, the term “an A₃AR ligand” includes one or more compounds which are capable of specifically affecting, directly or indirectly, fully or partially, the activity of the A₃AR.

Further, as used herein, the term “comprising” is intended to mean that the composition include the recited active agent, i.e. A₃AR ligand, but not excluding other elements, such as physiologically acceptable carriers and excipients as well as other active agents. The term “consisting essentially of” is used to define compositions which include the recited elements but exclude other elements that may have an essential significance on treatment of CRS. “Consisting of” shall thus mean excluding more than trace elements of other elements. Embodiments defined by each of these transition terms are within the scope of this invention.

Further, all numerical values, e.g. when referring the amounts or ranges of the elements constituting the composition comprising the A₃AR ligand as an active ingredient, are approximations which are varied (+) or (−) by up to 20%, at times by up to 10% of from the stated values. It is to be understood, even if not always explicitly stated that all numerical designations are preceded by the term “about”.

The invention will now be exemplified in the following description of experiments that were carried out in accordance with the invention. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways, than as specifically described hereinbelow.

### NON-LIMITING EXAMPLES

### Example 1: Clinical Study with the A₃AR Agonists: IB-MECA (CF101) and Cl-IB-MECA (CF102)

The clinical study (the “Study”) is a Randomized, Double-Masked, Placebo-Controlled, Parallel-Group Study of the Safety and Efficacy of CF101 (a pharmaceutical composition comprising IB-MECA as the active ingredient) and CF102. CF101 and CF102 are administered Orally in patients with moderate-to-severe immunotherapy associated CRS. Patients are randomized to receive pills of either CF101 or CF102 or matching placebo, given orally.

The patients that are enrolled in the study have to meet a number of inclusion criteria (namely criteria that had to be matched for a human subject to participate in the study), including (1) relapsing hematological cancer and any relapse following immunotherapy (2) adequate organ functioning.

Clinical outcome in improving CRS symptoms are determined through a variety of relevant parameters such as: the frequency of CRS, in particular grade 4 CRS (according to the grading system of the National Cancer Institute Common Terminology Criteria for Adverse Events (CTCAE v4.)), tumor response, duration of cancer emission, peak plasma concentration of the immunotherapy agent, frequency of medical interventions, inhibition of cytokine pathways, diminishing inflammatory cytokines, correlation between CF101 or CF102 density and inflammatory indications.

In addition, adverse events are recorded for each patient throughout the study and all effects reported by patients are reported.

1. A method of managing cytokine release syndrome (CRS) in a subject undergoing immunotherapy treatment, the method comprises administering to the subject an amount of an A₃ adenosine receptor (A₃AR) ligand effective to manage one or more of (i) level of at least one inflammatory cytokine and (ii) at least one CRS symptom; wherein said management is without significantly affecting said immunotherapy treatment.

2. The method of claim 1, wherein said administering comprises administration of the A₃AR before, concomitant or after said immunotherapy.

3. The method of claim 1 or 2, wherein said amount of the A₃AR ligand is effective to provide a statistically significant net change in the level of said at least one inflammatory cytokine as compared to a reference level of said at least one inflammatory cytokine.

4. The method of claim 3, wherein said reference level of the at least one inflammatory cytokine is the level of said at least one inflammatory cytokine in a peripheral blood sample of said subject obtained prior to administering of the A₃AR ligand.

5. The method of any one of claims 1 to 4, wherein said at least one inflammatory cytokine is selected from the group consisting of TNF-α, INF-7, IL-1, IL-6, IL-13, MIP1α, gp130, eotaxin and any combination of same.

6. The method of claim 5, wherein said at least one inflammatory cytokine is IL-6.

7. The method of any one of claims 1 to 6, wherein said at least one CRS symptom is selected from the group consisting of fatigue, fever, nausea, vomiting, headache, rash, diarrhea, tachypnea, hypoxemia, tachycardia, widened pulse pressure, hypotension, increased cardiac output, potentially diminished cardiac output, elevated D-dimer, hypofibrinogenemia, azotemia, transaminitis, hyperbilirubinemia, confusion, delirium, word finding difficulty, frank aphasia, hallucinations, tremor, dymetria, altered gait, seizures and combination of any of the above.

8. The method of any one of claims 1 to 7, wherein said immunotherapy is selected from Chimeric Antigen Receptor (CAR)-T cell Therapy, monoclonal antibody therapy and Mononuclear cell adoptive immunotherapy, anti-PD-1 therapy.

9. The method of claim 8, wherein said immunotherapy comprises CAR-T cell therapy.

10. The method of any one of claims 1 to 9, comprising administering to a subject being diagnosed with cancer.

11. The method of any one of claims 1 to 10, comprising administering to a subject being diagnosed with hematological cancer.

12. The method of claim 11, wherein said hematological cancer is cancer associated with CD19 expression, such as B-cell acute lymphocytic leukemia (B-ALL), T-cell acute lymphocytic leukemia (T-ALL), acute lymphocytic leukemia (ALL), chronic myelogenous leukemia (CML), chronic lymphocytic leukemia (CLL), B-cell promyelocytic leukemia, blastic plasmacytoid dendritic cell neoplasm, Burkitt's lymphoma, diffuse large B cell lymphoma, GC (germinal center)-DLBCL, NGC (non-germinal center) -DLBCL, transformed FL, double hit DLBCL, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, and Waldenstrom macroglobulinemia.

13. The method of claim 12, wherein said cancer is chronic lymphocytic leukemia.

14. The method of any one of claims 1 to 13, wherein said A₃AR ligand is selected from (i) A₃AR agonist; and (ii) A₃AR allosteric enhancer.

15. The method of claim 14, wherein said A₃AR agonist of formula (V) 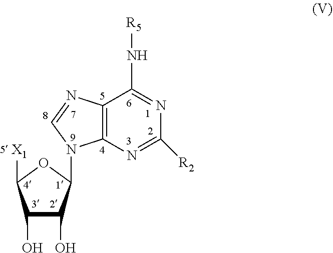
wherein
X₁ is R^{a}R^{b}NC(═O), wherein R^{a} and R^{b} may be the same or different and are selected from the group consisting of hydrogen, C₁-C₁₀ alkyl, amino, C₁-C₁₀ haloalkyl, C₁-C₁₀ aminoalkyl, and C₃-C₁₀ cycloalkyl;
R₂ is selected from the group consisting of hydrogen, halo, C₁-C₁₀ alkyoxy, amino, C₂-C₁₀ alkenyl, and C₂-C₁₀ alkynyl; and
R₅ is selected from the group consisting of R- and S-1-phenylethyl, an unsubstituted benzyl group, and a benzyl group substituted in one or more positions with a substituent selected from the group consisting of C₁-C₁₀ alkyl, amino, halo, C₁-C₁₀ haloalkyl, nitro, hydroxy, acetamido, C₁-C₁₀ alkoxy, and sulfo, and
pharmaceutically acceptable salts thereof.

16. The method of claim 15, wherein the A₃AR agonist selected from the group consisting of N⁶-2-(4-aminophenyl)ethyladenosine (APNEA), N⁶-(4-amino-3-iodobenzyl) adenosine-5′-(N-methyluronamide) (AB-MECA), N⁶-(3-iodobenzyl)-adenosine-5′-N-methyluronamide (IB-MECA) and 2-chloro-N⁶-(3-iodobenzyl)-adenosine-5′-N-methyluronamide (Cl-IB-MECA).

17. The method of claim 16, wherein the A₃AR agonist is IB-MECA.

18. The method of claim 14, wherein said A₃AR being A₃AR allosteric enhancer.

19. The method of claim 18, wherein said A₃AR allosteric enhancer is an imidazoquinoline derivative of formula (VII) 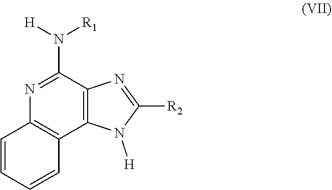
wherein:
R₁ represents an aryl or alkaryl being optionally substituted at the aromatic ring with one or more substituents selected from the group consisting of C₁-C₁₀ alkyl, halo, C₁-C₁₀ alkanol, hydroxyl, C₁-C₁₀ acyl, C₁-C₁₀ alkoxyl, C₁-C₁₀-alkoxycarbony, C₁-C₁₀ alkoxylalkyl, C₁-C₁₀ thioalkoxy, C₁-C₁₀ alkylether, amino, hydrazido, C₁-C₁₀ alkylamino, pyridylthio, C₂-C₁₀ alkenyl, C₂-C₁₀ alkynyl, thio, C₁-C₁₀ alkylthio, acetoamido, and sulfonic acid; or said substituents can form together a cycloalkyl or cycloalkenyl fused to said aryl, the cycloalkyl or cycloalkenyl optionally comprising one or more heteroatoms; provided that said aryl is not an unsubstituted phenyl group;
R₂′ represents hydrogen or a substituent selected from the group consisting of C₁-C₁₀ alkyl, C₂-C₁₀ alkenyl, C₂-C₁₀ alkynyl, C₄-C₁₀ cycloalkyl, C₄-C₁₀ cycloalkenyl, a five to seven membered heterocyclic aromatic ring, C₅-C₁₅ fused cycloalkyl, bicyclic aromatic or heteroaromatic rings, C₁-C₁₀ alkylether, amino, hydrazido, C₁-C₁₀ alkylamino, C₁-C₁₀ alkoxy, C₁-C₁₀-alkoxycarbony, C₁-C₁₀ alkanol, C₁-C₁₀ acyl, C₁-C₁₀ thioalkoxy, pyridylthio, thio, C₁-C₁₀ alkylthio, acetoamido, and sulfonic acid;
and pharmaceutically acceptable salts thereof.

20. The method of claim 19, wherein said A₃AR allosteric enhancer is selected from the group consisting of:
N-(3,4-Dichloro-phenyl)-2-cyclopentyl-1H-imidazo[4,5-c]quinolin-4-amine;
N-(3,4-Dichloro-phenyl)-2-cycloheptyl-1H-imidazo[4,5-c]quinolin-4-amine;
N-(3,4-Dichloro-phenyl)-2-cyclobutyl-1H-imidazo[4,5-c]quinolin-4-amine; and
N-(3,4-Dichloro-phenyl)-2-cyclohexyl-1H-imidazo[4,5-c]quinolin-4-amine.

21. The method of claim 20, wherein said imidazoquinoline derivative is N-(3,4-Dichloro-phenyl)-2-cyclohexyl-1H-imidazo[4,5-c]quinolin-4-amine.

22. An A₃ adenosine receptor (A₃AR) ligand for use in the management of cytokine release syndrome (CRS) in a subject undergoing immunotherapy treatment, said management comprises one or more of (i) managing level of at least one inflammatory cytokine and (ii) managing at least one CRS symptom; wherein said management is without significantly affecting said immunotherapy treatment.

23. The A₃AR ligand for use as claimed in claim 22, wherein said management comprises management, without significantly affecting said immunotherapy treatment, of one or more of (i) managing level of at least one inflammatory cytokine and (ii) managing at least one CRS symptom.

24. The A₃AR ligand for use as claimed in claim 22, before, concomitant or after said immunotherapy.

25. The A₃AR ligand for use as claimed in any one of claims 22 to 24, in an amount effective to provide a statistically significant net change in the level of said at least one inflammatory cytokine as compared to a reference level of said at least one inflammatory cytokine.

26. The A₃AR ligand for use as claimed in claim 25, wherein said reference level of the at least one inflammatory cytokine is the level of said at least one inflammatory cytokine in a peripheral blood sample of said subject obtained prior to the use of the A₃AR ligand.

27. The A₃AR ligand for use as claimed in any one of claims 22 to 26, wherein said at least one inflammatory cytokine is selected from the group consisting of TNF-α, INF-γ, IL-1, IL-6, IL-13, MIP1α, gp130, eotaxin and any combination of same.

28. The A₃AR ligand for use as claimed in claim 27, wherein said at least one inflammatory cytokine is IL-6.

29. The A₃AR ligand for use as claimed any one of claims 22 to 28, wherein said at least one CRS symptom is selected from the group consisting of fatigue, fever, nausea, vomiting, headache, rash, diarrhea, tachypnea, hypoxemia, tachycardia, widened pulse pressure, hypotension, increased cardiac output, potentially diminished cardiac output, elevated D-dimer, hypofibrinogenemia, azotemia, transaminitis, hyperbilirubinemia, confusion, delirium, word finding difficulty, frank aphasia, hallucinations, tremor, dymetria, altered gait, seizures and combination of any of the above.

30. The A₃AR ligand for use as claimed in any one of claims 22 to 29, wherein said immunotherapy is selected from Chimeric Antigen Receptor (CAR)-T cell Therapy, monoclonal antibody therapy and Mononuclear cell adoptive immunotherapy, anti-PD-1 therapy.

31. The A₃AR ligand for use as claimed in claim 30, wherein said immunotherapy comprises CAR-T cell therapy.

32. The A₃AR ligand for use as claimed in any one of claims 22 to 31, wherein said subject is diagnosed with cancer.

33. The A₃AR ligand for use as claimed in any one of claims 22 to 32, wherein said subject is diagnosed hematological cancer.

34. The A₃AR ligand for use as claimed in claim 33, wherein said subject is diagnosed claim 11, wherein said hematological cancer is cancer associated with CD19 expression, such as B-cell acute lymphocytic leukemia (B-ALL), T-cell acute lymphocytic leukemia (T-ALL), acute lymphocytic leukemia (ALL), chronic myelogenous leukemia (CML), chronic lymphocytic leukemia (CLL), B-cell promyelocytic leukemia, blastic plasmacytoid dendritic cell neoplasm, Burkitt's lymphoma, diffuse large B cell lymphoma, GC (germinal center)-DLBCL, NGC (non-germinal center) -DLBCL, transformed FL, double hit DLBCL, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, and Waldenstrom macroglobulinemia.

35. The A₃AR ligand for use as claimed in claim 34, wherein said subject is diagnosed, wherein said cancer is chronic lymphocytic leukemia.

36. The A₃AR ligand for use as claimed in any one of claims 22 to 31, wherein said A₃AR ligand is selected from (i) A₃AR agonist; and (ii) A₃AR allosteric enhancer.

37. The A₃AR ligand for use as claimed in claim 36, wherein said A₃AR agonist of formula (V) 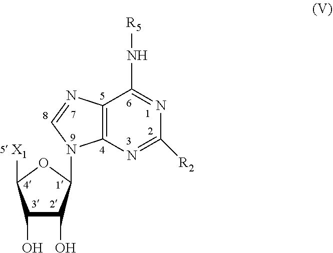
wherein
X₁ is R^{a}R^{b}NC(═O), wherein R^{a} and R^{b} may be the same or different and are selected from the group consisting of hydrogen, C₁-C₁₀ alkyl, amino, C₁-C₁₀ haloalkyl, C₁-C₁₀ aminoalkyl, and C₃-C₁₀ cycloalkyl;
R₂ is selected from the group consisting of hydrogen, halo, C₁-C₁₀ alkyoxy, amino, C₂-C₁₀ alkenyl, and C₂-C₁₀ alkynyl; and
R₅ is selected from the group consisting of R- and S-1-phenylethyl, an unsubstituted benzyl group, and a benzyl group substituted in one or more positions with a substituent selected from the group consisting of C₁-C₁₀ alkyl, amino, halo, C₁-C₁₀ haloalkyl, nitro, hydroxy, acetamido, C₁-C₁₀ alkoxy, and sulfo, and
pharmaceutically acceptable salts thereof.

38. The A₃AR ligand for use as claimed in claim 37, wherein the A₃AR agonist selected from the group consisting of N⁶-2-(4-aminophenyl)ethyladenosine (APNEA), N⁶-(4-amino-3-iodobenzyl) adenosine-5′-(N-methyluronamide) (AB-MECA), N⁶-(3-iodobenzyl)-adenosine-5′-N-methyluronamide (IB-MECA) and 2-chloro-N⁶-(3-iodobenzyl)-adenosine-5′-N-methyluronamide (Cl-IB-MECA).

39. The A₃AR ligand for use as claimed in claim 38, wherein the A₃AR agonist is IB-MECA.

40. The A₃AR ligand for use as claimed in claim 36, wherein said A₃AR being A₃AR allosteric enhancer.

41. The A₃AR ligand for use as claimed in claim 40, wherein said A₃AR allosteric enhancer is an imidazoquinoline derivative of formula (VII) 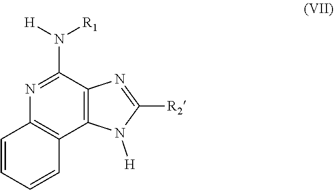
wherein:
R₁ represents an aryl or alkaryl being optionally substituted at the aromatic ring with one or more substituents selected from the group consisting of C₁-C₁₀ alkyl, halo, C₁-C₁₀ alkanol, hydroxyl, C₁-C₁₀ acyl, C₁-C₁₀ alkoxyl, C₁-C₁₀-alkoxycarbony, C₁-C₁₀ alkoxylalkyl, C₁-C₁₀ thioalkoxy, C₁-C₁₀ alkylether, amino, hydrazido, C₁-C₁₀ alkylamino, pyridylthio, C₂-C₁₀ alkenyl, C₂-C₁₀ alkynyl, thio, C₁-C₁₀ alkylthio, acetoamido, and sulfonic acid; or said substituents can form together a cycloalkyl or cycloalkenyl fused to said aryl, the cycloalkyl or cycloalkenyl optionally comprising one or more heteroatoms; provided that said aryl is not an unsubstituted phenyl group;
R₂′ represents hydrogen or a substituent selected from the group consisting of C₁-C₁₀ alkyl, C₂-C₁₀ alkenyl, C₂-C₁₀ alkynyl, C₄-C₁₀ cycloalkyl, C₄-C₁₀ cycloalkenyl, a five to seven membered heterocyclic aromatic ring, C₅-C₁₅ fused cycloalkyl, bicyclic aromatic or heteroaromatic rings, C₁-C₁₀ alkylether, amino, hydrazido, C₁-C₁₀ alkylamino, C₁-C₁₀ alkoxy, C₁-C₁₀-alkoxycarbony, C₁-C₁₀ alkanol, C₁-C₁₀ acyl, C₁-C₁₀ thioalkoxy, pyridylthio, thio, C₁-C₁₀ alkylthio, acetoamido, and sulfonic acid;
and pharmaceutically acceptable salts thereof.

42. The A₃AR ligand for use as claimed in claim 41, wherein said A₃AR allosteric enhancer is selected from the group consisting of:
N-(3,4-Dichloro-phenyl)-2-cyclopentyl-1H-imidazo[4,5-c]quinolin-4-amine;
N-(3,4-Dichloro-phenyl)-2-cycloheptyl-1H-imidazo[4,5-c]quinolin-4-amine;
N-(3,4-Dichloro-phenyl)-2-cyclobutyl-1H-imidazo[4,5-c]quinolin-4-amine; and
N-(3,4-Dichloro-phenyl)-2-cyclohexyl-1H-imidazo[4,5-c]quinolin-4-amine.

43. The A₃AR ligand for use as claimed in claim 42, wherein said imidazoquinoline derivative is N-(3,4-Dichloro-phenyl)-2-cyclohexyl-1H-imidazo[4,5-c]quinolin-4-amine.

44. A pharmaceutical composition for the management of cytokine release syndrome (CRS) in a subject undergoing immunotherapy treatment, the composition comprising a physiologically acceptable carrier and an a therapeutically effective amount of A₃ adenosine receptor (A₃AR) ligand.

45. The pharmaceutical composition of claim 44, wherein said A₃AR ligand is for use as claimed in any one of claims 22 to 44.

46. The pharmaceutical composition of claim 45, in a dosage form for oral administration.
